(19) United States
(12) Patent Application Publication
(10) Pub. No.: US 2008/0045761 A1
(43) Pub. Date: Feb. 21, 2008

(54) METHOD AND APPARATUS FOR STEAM DEALKYLATION IN A PLANT FOR THE CATALYTIC REFORMING OF HYDROCARBONS

(75) Inventors: Helmut Fritz, Muenchen (DE); Volker Goeke, Wolfratshausen (DE)

Correspondence Address: CROWELL & MORING LLP;INTELLECTUAL PROPERTY GROUP, P.O. BOX 14300, WASHINGTON, DC, 20044-4300, US

(73) Assignee: Linde Aktiengesellschaft, Wiesbaden (DE)

(21) Appl. No.: 11/840,904

(22) Filed: Aug. 17, 2007

(30) Foreign Application Priority Data
Aug. 18, 2006 (DE) 10 2006 038 889.5
Dec. 12, 2006 (DE) 10 2006 058 532.1

(51) Int. Cl. C07C 13/20

(52) U.S. Cl. 585/15; 422/202; 585/402; 585/410; 585/487

(57) ABSTRACT

A method and apparatus for treating a fraction consisting predominantly of hydrocarbons having at least seven carbon atoms (C₇₊ fraction) as produced in a plant for catalytic reforming of hydrocarbon-containing feedstock, is disclosed. Following hydration, the C₇₊ fraction is taken to steam dealkylation where the useable products benzene and hydrogen are produced.

This application claims the priority of German Patent Documents No. 10 2006 038 889.5, filed Aug. 18, 2006, and No. 10 2006 058 532.1, filed Dec. 12, 2006, the disclosures of which are expressly incorporated by reference herein.

### BACKGROUND AND SUMMARY OF THE INVENTION

The invention relates to a method for treating a fraction consisting predominantly of hydrocarbons having at least seven carbon atoms (C₇₊ fraction) as produced in a plant for the catalytic reforming of hydrocarbon-containing feedstock, and also an apparatus for carrying out the method.

Heavy naphtha is produced primarily in a plant for the catalytic reforming of hydrocarbon-containing feedstock, such as is produced, for example, in crude oil distillation.

The heavier naphtha, such as is produced in crude oil distillation, contains primarily iso- and n-paraffins, napthenes and aromatics containing primarily six to twelve carbon atoms, where the percentage of aromatics may be very small and is dependent on the feedstock. In accordance with the prior art, the heavier naphtha first undergoes desulfurization involving the consumption of hydrogen and the creation of hydrogen sulfide and then conducted to catalytic reformation as feedstock. In catalytic reformation, the existing paraffins and napthenes are converted into aromatics in the presence of a catalyst, where hydrogen and light hydrocarbons are produced as by-products. These by-products are separated from the reaction products from the catalytic reformation, so that a fraction consisting predominantly of hydrogen and hydrocarbons having up to five carbon atoms and a fraction consisting predominantly of hydrocarbons having a fraction consisting predominantly of hydrocarbons having at least six carbon atoms (C₆₊ fraction) is produced. This C₆₊ fraction contains aromatics as an economically usable product, principally benzene, which find an application as the feedstock for the synthesis of numerous plastic materials and to increase the knock resistance of gasoline.

In order to acquire the economically viable products from the C₆₊ fraction, primarily benzene, and to maximize the yield, the following method is used in accordance with the prior art. The C₆₊ fraction is separated into a fraction consisting predominantly of hydrocarbons having six carbon atoms (C₆₊ fraction) and a fraction consisting predominantly of hydrocarbons having at least seven carbon atoms (C₇₊ fraction). The economically viable product benzene can be separated directly from the C₆₊ fraction. By means of fluid-fluid extraction, the linear hydrocarbons can be separate from the C₇₊ fraction and further processed as a raffinate, the raffinate can be returned to the feedstock for catalytic reforming. The C₇₊ fraction freed from the linear hydrocarbons now contains primarily aromatics having seven to eight carbon atoms and is separated into a fraction consisting predominantly of hydrocarbons having seven carbon atoms (primarily toluene) and into a fraction consisting predominantly of hydrocarbons having eight carbon atoms (primarily xylene). The fraction consisting predominantly of hydrocarbons having seven carbon atoms is taken as feedstock material to a method for hydro-dealkylation.

A method of this type for hydro-dealkylation is described, for example, in WO2005071045. The hydrocarbons are contacted with hydrogen in the presence of a catalyst at a temperature of 400° C. to 650° C. and at a pressure between 20 bar and 40 bar, where the hydrogen is present at a molar excess of three to six times the hydrocarbons. Under these conditions the alkyl groups are split off from the specific alkylated aromatics (for example, toluene or xylene) so that benzene and the specific alkanes (for example, methane and ethane) form.

The consumption of hydrogen in the hydro-dealkylation of the hydrocarbons has a negative economic effect on this method from the prior art for extracting benzene.

### BRIEF DESCRIPTION OF THE DRAWING

FIG. 1 illustrates an embodiment of an apparatus in accordance with the principles of the present invention.

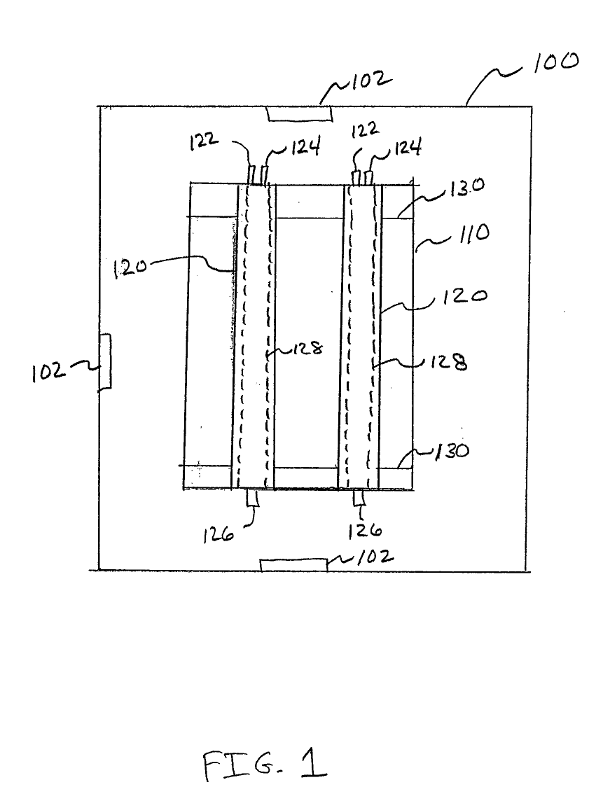

### DETAILED DESCRIPTION OF THE EXEMPLARY EMBODIMENTS

In accordance with the invention, with respect to the method, the C₇₊ fraction is subjected to steam dealkylation where mainly the two utilizable products benzene and hydrogen are produced along with reaction products such as carbon monoxide and carbon dioxide.

The basic idea of the invention is to carry out the dealkylation of the alkylated aromatics while generating benzene with the aid of steam dealkylation. Steam dealkylation requires only inexpensive steam as the starting material and produces the valuable by-product hydrogen in addition to the desired quality product benzene.

The C₇₊ fraction employed in the steam dealkylation contains primarily:
a) aromatic hydrocarbons having seven to ten carbon atoms,
b) cyclic paraffins (cycloalkenes) having six to ten carbon atoms,
c) iso- and n-paraffins having six to ten carbon atoms,
d) alkenes having seven to ten carbon atoms, or
   any mixture of the preceding, in which the exact composition of the mixture depends on the composition of the heavier naphtha which is taken as feedstock for catalytic reforming. The method in accordance with the invention is suitable for each of the compounds of the C₇₊ fractions described.

The hydrocarbons from the C₇₊ fraction react advantageously with steam in the gas phase with the introduction of heat on a solid catalyst. The gaseous C₇₊ fraction is dealkylated by the presence of gaseous water (steam) on a catalyst under the constant introduction of heat, whereby the desired products benzene and hydrogen are produced in addition to carbon monoxide, carbon dioxide and additional by-products.

Preferably the heat required for the dealkylation reaction is generated from combustion of a starting material with air. It proves to be particularly advantageous to use gaseous reaction by-products from the steam dealkylation, specifically carbon monoxide and methane as the starting material for combustion with air. A part of the gaseous reaction products from the steam dealkylation, in particular carbon monoxide and methane, is combustible and can thus serve as starting material for combustion to generate the required reaction heat. This saves heating gas and this otherwise unused part of the reaction products is employed usefully.

Following compression, the gaseous reaction products are expediently separated by way of pressure swing adsorption into gaseous hydrogen and gaseous reaction by-products, specifically carbon monoxide, carbon dioxide and methane. The valuable by-product hydrogen is also present in gaseous form and can be employed much more usefully than in combustion. By means of pressure swing adsorption preceded by compression, the hydrogen can easily be separated from the combustible gaseous reaction by-products which can serve as starting material in the combustion.

Advantageously the flue gases generated in the combustion are cooled via a heat exchanger while heating the starting materials for the steam dealkylation. By using the heat from the flue gases to preheat the starting materials (C₇₊ fraction and steam) for the steam dealkylation, the necessary heat which has to be brought in to maintain the required temperatures for the dealkylation reaction is reduced. This achieves an economical use of energy resources.

The C₇₊ fraction and the steam are advantageously taken past the solid catalyst in pipes, preferably from top to bottom, with the catalyst being located inside the pipes. Heat is expediently brought to the pipes from the outside. The heat required for the dealkylation reaction is advantageously transferred to the pipe by electromagnetic radiation, thermal radiation and/or convection. The actual dealkylation reaction takes place inside the pipes where the catalyst is located. The two components in the reaction (C₇₊ fraction and steam) are taken from top to bottom through the pipes filled with the catalyst. The heat required for the dealkylation reaction is generated outside the pipes and transferred to the pipes by the mechanisms named from where the heat is transferred by means of conduction and convection into the interior of the pipes where the reaction is taking place.

Preferably a solid catalyst of a porous carrier material is used, in particular γ-Al₂O₃, MgAl spinel and/or Cr₂O₃, and an active component on the surface of the carrier material, in particular Rh with 0.1-1.0% loading by weight and/or Pd with 0.2-2.0% loading by weight.

The steam dealkylation is advantageously performed at a temperature of 400° C. to 600° C., preferably 450° C. to 550° C., particularly preferably 480° C. to 520° C. and at a pressure of 1 to 15 bar, preferably 1.2 to 10 bar, particularly preferably 1.5 to 8 bar.

The steam dealkylation is expediently performed at a molar quotient of steam to hydrocarbons which lies in the range from 1 to 20, preferably from 2 to 15, when it enters the reactor. In another embodiment of the invention, the steam dealkylation is performed at a molar quotient of steam to hydrocarbons which lies in the range from 3 to 12, preferably from 5 to 10 when it enters the reactor. Generally the steam dealkylation is performed with a molar excess of water, where the exact ratio in the different embodiments of the invention depends on the precise composition of the C₇₊ fraction.

It proves advantageous to subject the C₇₊ fraction before steam dealkylation to a process to convert dienes and styrenes, where specifically hydrating methods consuming hydrogen are employed. In another embodiment of the invention, the C₇₊ fraction is separated before steam dealkylation from a fraction of hydrocarbons having at least six carbon atoms where the fraction of hydrocarbons having at least six carbon atoms is subjected to a process to convert dienes and styrenes, specifically a hydrating process which consumes hydrogen. By employing the hydrating methods, any diolefins present in the C₇₊ fraction are converted into their corresponding olefins, just as components containing sulfur, nitrogen and oxygen can be converted and removed. Deactivation of the catalyst is reduced and the life of the catalyst is clearly increased.

The reaction products from the steam dealkylation are preferably cooled and separated in a 3-phase separation into gaseous reaction products, hydrocarbons and water. The reaction products coming from the steam dealkylation contain not only the desired quality products benzene and hydrogen but also reaction products such as carbon monoxide and carbon dioxide and reaction by-products. To obtain the desired quality products, the reaction products must be separated. This is done by way of a 3-phase separation of the cooled reaction products into the gaseous reaction products, in particular hydrogen, carbon monoxide, carbon dioxide and methane, into hydrocarbons, in particular benzene, and into water.

The hydrogen generated in the steam dealkylation of the C₇₊ fraction is expediently fed completely or partially into the starting material for the hydrogen-consuming processes. The hydrogen generated in the steam dealkylation can be used entirely or partially for the hydrogen-consuming processes described in the previous section so that the need for hydrogen to be supplied externally is minimized.

In one embodiment of the invention, the hydrogen generated in the steam dealkylation of the C₇₊ fraction is taken as the starting material to any number of other hydrogen-consuming processes in the oil refinery, preferably to a process for the conversion and removal of sulfur-containing components or to a process for reforming hydrocarbon-containing feedstock by means of hydrogen.

Reduction of the sulfur content in the C₇₊ fraction to below 10 ppm, preferably to below 3 ppm, particularly preferably to below 1 ppm, before steam dealkylation proves advantageous for a good yield of the desired reaction product benzene.

Preferably the benzene is separated from the hydrocarbons of the reaction products through rectification. Following rectification, the benzene advantageously undergoes adsorptive fine cleaning to dry and remove the trace components, where the benzene is directed across an adsorbent on which the trace components, as opposed to benzene, are adsorbed. By applying the inventive method, the benzene can be extracted from the reaction products by simple rectification and processed further or marketed. Expensive extraction or extractive rectification as when applying a process in accordance with the prior art is not necessary, thus reducing investment and process costs.

Components boiling close to benzene or components forming azeotropes in the C₇₊ fraction are advantageously converted by the steam dealkylation. All reaction products boiling heavier than benzene from rectification, consisting predominantly of non-converted feedstock from the steam deakylation are expediently returned to steam dealkylation through optional hydration as feedstock. In another embodiment of the invention, all reaction products boiling heavier than benzene from rectification, consisting predominantly of non-converted feedstocks from steam dealkylation are returned for hydration of the C₇₊ fraction, a C₆₊ fraction or for hydration of a fraction consisting predominantly of hydrocarbons having at least five carbon atoms prior to steam dealkylation. By returning the non-converted feedstock for hydration or for steam dealkylation, circulation is achieved without losing valuable feedstocks.

In another embodiment of the invention, prior to steam dealkylation a fraction consisting predominantly of hydrocarbons having at least eight carbon atoms (C₈₊ fraction) is separated by distillation from the C₇₊ fraction, where the separated C₈₊ fraction is taken to a process for extracting paraxylene.

Concerning the apparatus, the object of the invention is achieved by the apparatus comprising an oven 100 with a furnace 110 and pipes 120 located in the furnace. The actual steam dealkylation takes place in the pipes which in turn are located in the furnace of the oven where the heat required for steam dealkylation can be generated.

The pipes are advantageously installed vertically in the furnace and have heat expansion compensating elements 130 at the lower and/or upper end. The heat expansion compensating elements at the lower and/or upper end of the vertical pipes prevent mechanical stress from temperature differences which can lead to increased wear of the pipes.

Each pipe expediently has a supply for the C₇₊ fraction and the steam, 122, 124, respectively, and an outlet 126 for the reaction products.

It similarly proves advantageous that each pipe is filled on the inside with a catalyst 128, where the catalyst consists of a porous carrier material, in particular γ-Al₂O₃, MgAl spinel and/or Cr₂O₃ and an active component on the surface of the carrier material, in particular Rh with 0.1-1.0% loading by weight and/or Pd with 0.2-2.0% loading by weight.

Preferably the oven has at least one burner 102 on the wall, the ceiling and/or the floor. The pipes are expediently suitable for an internal pressure of 1 to 15 bar, preferably 1.2 to 10 bar, particularly preferably 1.5 to 8 bar, and for use in an oven with flame temperatures of up to 1400° C.

The present invention is successful specifically in creating an economical alternative to the prior art for treating a C₇₊ fraction. Through the application of the inventive method and the inventive apparatus, the valuable by-product hydrogen is generated in addition to the usable product benzene.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for treating a fraction consisting predominantly of hydrocarbons having at least seven carbon atoms (C₇₊ fraction) as produced in a plant for catalytic reforming of hydrocarbon-containing feedstock, wherein the C₇₊ fraction undergoes steam dealkylation, where two usable product materials benzene and hydrogen are produced in addition to reaction products such as carbon monoxide and carbon dioxide.

2. The method according to claim 1, wherein the C₇₊ fraction contains:
a) aromatic hydrocarbons having seven to ten carbon atoms;
b) cyclic paraffins (cycloalkenes) having six to ten carbon atoms;
c) iso- and n-paraffins having six to ten carbon atoms;
d) alkenes having seven to ten carbon atoms; or
any mixture of the aforementioned.

3. The method according to claim 1, wherein the hydrocarbons from the C₇₊ fraction react with water in a gas phase with addition of heat to a solid catalyst.

4. The method according to claim 1, wherein heat required for the dealkylation reaction is generated by combustion of a starting material with air.

5. The method according to claim 1, wherein gaseous reaction products from the steam dealkylation are separated following compression by way of pressure swing adsorption into gaseous hydrogen and gaseous reaction by-products, specifically carbon monoxide, carbon dioxide and methane.

6. The method according to claim 5, wherein the gaseous reaction by-products from the steam dealkylation, specifically carbon monoxide and methane, are used as starting material for the combustion with air.

7. The method according to claim 1, wherein flue gases generated during combustion are cooled by a heat exchanger while heating starting materials for the steam dealkylation.

8. The method according to claim 1, wherein the C₇₊ fraction and the steam are conducted in pipes, from top to bottom, past a solid catalyst, where the catalyst is on an inside of the pipes.

9. The method according to claim 8, wherein heat is brought to the pipes from outside.

10. The method according to claim 9, wherein the heat required for the dealkylation reaction is transferred to the pipes by electromagnetic radiation, thermal radiation and/or convection.

11. The method according to claim 1, wherein a solid catalyst of a porous carrier material is used, specifically γ-Al₂O₃, MgAl spinel and/or Cr₂O₃ and an active component on a surface of the carrier material, in particular Rh with 0.1-1.0% loading by weight and/or Pd with 0.2-2.0% loading by weight.

12. The method according to claim 1, wherein the steam dealkylation is performed at a temperature of 400° C. to 600° C., preferably 450° C. to 550° C., particularly preferably 480° C. to 520° C.

13. The method according to claim 1, wherein the steam dealkylation is performed at a pressure from 1 to 15 bar, preferably 1.2 to 10 bar, particularly preferably 1.5 to 8 bar.

14. The method according to claim 1, wherein the steam dealkylation is performed at a molar quotient of steam to hydrocarbons in a range from 1 to 20, preferably from 2 to 15, when it enters a reactor.

15. The method according to claim 1, wherein the steam dealkylation is performed at a molar quotient of steam to hydrocarbons which is in a range from 3 to 12, preferably from 5 to 10, when it enters a reactor.

16. The method according to claim 1, wherein the C₇₊ fraction undergoes a process prior to the steam dealkylation to convert dienes and styrenes where in particular hydrating methods are employed involving consumption of hydrogen.

17. The method according to claim 1, wherein the C₇₊ fraction undergoes a process prior to the steam dealkylation to convert and to remove components containing sulfur, nitrogen and/or oxygen, in which specifically hydrating processes involving consumption of hydrogen are employed.

18. The method according to claim 1, wherein the reaction products from the steam dealkylation are cooled and separated into gaseous reaction products, hydrocarbons and water in a 3-phase separation.

19. The method according to claim 16, wherein the hydrogen produced in the steam dealkylation of the C₇₊ fraction is fed completely or partially into a starting material for the processes involving the consumption of hydrogen.

20. The method according to claim 17, wherein the hydrogen produced in the steam dealkylation of the C₇₊ fraction is fed completely or partially into a starting material for the processes involving the consumption of hydrogen.

21. The method according to claim 1, wherein the hydrogen produced in the steam dealkylation of the C₇₊ fraction is fed as starting material to a process consuming hydrogen in an oil refinery, preferably into a process to convert and remove components containing sulfur or a process to split hydrocarbon-containing starting material via hydrogen.

22. The method according to claim 1, wherein a sulfur content in the C₇₊ fraction is reduced to below 10 ppm, preferably below 3 ppm, particularly preferably below 1 ppm prior to the steam dealkylation.

23. The method according to claim 1, wherein the benzene is separated from the hydrocarbons by way of rectification of the reaction products.

24. The method according to claim 23, wherein the benzene undergoes adsorptive fine cleaning following rectification to dry and remove trace components, where the benzene is passed across an adsorbent on which the trace components are adsorbed.

25. The method according to claim 1, wherein components boiling close to benzene or forming azeotropes in the C₇₊ fraction are converted by steam dealkylation.

26. The method according to claim 23, wherein all heavier boiling reaction products than benzene from rectification, consisting predominantly of non-converted feedstocks from the steam dealkylation are returned to the steam dealkylation as feedstock via optional hydration.

27. The method according to claim 23, wherein all heavier boiling reaction products than benzene from rectification consisting predominantly of non-converted feedstocks from the steam dealkylation are returned prior to steam dealkylation for hydration of the C₇₊ fraction, a C₆₊ fraction or for hydration of a fraction consisting predominantly of hydrocarbons having at least five carbon atoms.

28. The method according to claim 1, wherein prior to steam dealkylation a fraction consisting predominantly of hydrocarbons having at least eight carbon atoms (C₈₊ fraction) is separated though distillation from the C₇₊ fraction as feedstock, where the separated C₈₊ fraction is taken as feedstock to a process to extract paraxylene.

29. An apparatus for treating a fraction consisting predominantly of hydrocarbons having at least seven carbon atoms (C₇₊ fraction) as produced in a plant for catalytic reforming of hydrocarbon-containing feedstock, wherein the apparatus includes an oven with a furnace and pipes located in the furnace.

30. The apparatus according to claim 29, wherein the pipes are mounted vertically in the furnace and have heat expansion compensating elements at a lower and/or an upper end.

31. The apparatus according to claim 29, wherein each pipe has a supply for the C₇₊ fraction and the steam and an outlet for the reaction products.

32. The apparatus according to claim 29, wherein each pipe is filled on an inside with a catalyst, where the catalyst consists of a porous carrier material, specifically γ-Al₂O₃, MgAl spinel and/or Cr₂O₃ and an active component on a surface of the carrier material, in particular Rh with 0.1-1.0% loading by weight and/or Pd with 0.2-2.0% loading by weight.

33. The apparatus according to claim 29, wherein the oven has at least one burner on a wall, a ceiling and/or a floor.

34. The apparatus according to claim 29, wherein the pipes are suitable for an internal pressure of 1 to 15 bar, preferably 1.2 to 10 bar, particularly preferably 1.5 to 8 bar, and for use in an oven with flame temperatures of up to 1400° C.

35. A method of extracting benzene from a hydrocarbon having at least seven carbon atoms, comprising the steps of:
producing the hydrocarbon having at least seven carbon atoms in a plant for catalytic reforming of hydrocarbon-containing feedstock;
subjecting the hydrocarbon having at least seven carbon atoms to steam dealkylation; and
producing benzene from the steam dealkylation.

36. The method according to claim 35, further comprising the step of producing hydrogen from the steam dealkylation.
